(19) United States
(12) Patent Application Publication
(10) Pub. No.: US 2023/0383281 A1
(43) Pub. Date: Nov. 30, 2023

(54) FILTRATION METHODS AND DEVICES FOR FAST NUCLEIC ACID EXTRACTION

(71) Applicant: James Jianmin Bao, San Ramon, CA (US)

(72) Inventor: James Jianmin Bao, San Ramon, CA (US)

(73) Assignee: Biomics Inc., San Ramon, CA (US)

(21) Appl. No.: 17/827,753

(22) Filed: May 29, 2022

(51) Int. Cl. C12N 15/10; C12Q 1/686

(52) U.S. Cl. CPC C12N 15/1017; C12N 15/1006; C12Q 1/686

(57) ABSTRACT

A membrane employing solid sorbents embedded within polymeric adhesives. The membrane was used to make filtration devices. Methods were developed based on the use of those devices for fast purification of nucleic acids, especially for the recovery of low molecular weight nucleic acids. Those devices offer the advantages of automation, high speed, high throughput, and suitability for point-of-care testing. They can be used either independently or combined with other conventional devices such as magnetic beads or spin columns.

### CROSS-REFERENCE TO RELATED APPLICATIONS

This application claims priority to U.S. provisional patent application No. 63/195,724 filed 2 June 2021; the disclosure of which is incorporated herein by reference in its entirety.

### FIELD OF THE INVENTION

This invention relates to a new system and method for nucleic acid (NA, DNA and/or RNA) purification. More specifically, it relates to a simple and rapid system and method for the filtration and purification of NA from cells.

### BACKGROUND OF THE INVENTION

Nucleic acid analysis is widely used in many clinic and genomic related diagnoses. With the proliferation of studies involving nucleic acids, laboratory procedures such as the polymerization chain reaction (PCR) and DNA sequencing have gained widespread use [1]. For all of those applications, NA has to be first extracted from biological cells and the quality of the extracted NA plays an important role. Actually, isolation of high quantities of pure, intact, double stranded, highly concentrated, not contaminated genomic DNA is prerequisite for successful and reliable large scale genotyping analysis.

In addition to providing purified nucleic acids for use in downstream applications, it is also important to ensure that total nucleic acids are isolated from a sample. This is particularly important for the isolation of RNA that may be used for studies involving gene expression and gene regulation, as the quantity of a specific RNA within a cell indicates the level of expression of a particular DNA. In recent years, the study of gene expression has increased, with gene activity and nucleic acids obtained from biological samples being used to diagnose infections or diseases including cancer, and to monitor the effects of administered drugs, among other applications. Information relating to the presence and quantity of a specific RNA is critical in studying gene expression; therefore, it is critical that the method of nucleic acid isolation being employed does not favor the isolation of certain sizes of RNA molecules.

Even further, with the spread of Covid-19 pandemic, there is an urgent demand for fast, convenient, sustainable, and reliable methods for rapid analyses and confirmation of the disease.

There are two classes of DNA that can be extracted from biological samples: 1) recombinant DNA constructs such as plasmids or bacteriophage; and 2) chromosomal or genomic DNA from prokaryotic or eukaryotic organisms [2]. In addition, there are some DNA fragments that are floating in biological liquid such as blood or urine. Those are called cell free DNA (cfDNA) and are very useful for some diagnosis.

On the other hand, RNA is an unstable molecule and has a very short half-life once extracted from the cell or tissues [3]. There are several types of naturally occurring RNA including ribosomal RNA (rRNA) (80%-90%), messenger RNA (mRNA) (2.5%-5%) and transfer RNA (tRNA) [4]. Special care and precautions are required for RNA isolation as it is susceptible to degradation [3, 5]. RNA is especially unstable due to the ubiquitous presence of RNases which are enzymes present in blood, all tissues, as well as most bacteria and fungi in the environment [3, 6]. Strong denaturants have always been used in intact RNA isolation to inhibit endogenous RNases [2]. RNA extraction relies on good laboratory technique and RNase-free technique. RNAse is heat-stable and refolds following heat denaturation. They are difficult to inactivate as they do not require cofactors [2]. The most common isolation methods can be divided into two classes: utilization of 4 M guanidinium thiocyanate and utilization of phenol and sodium dodecyl sulfate (SDS) [2].

Due to its complexity, the extraction of NA becomes the most crucial method in molecular biology and is becoming one of the limiting factors in use of the rapidly developed automation procedures. Generally, successful extraction and purification of NA require four major steps: 1) effective disruption of cells or tissue in cell lysis; 2) denaturation of nucleoprotein complexes; 3) inactivation of nucleases, for example, RNase for RNA extraction and DNase for DNA extraction; and 4) away from contamination and cell debris [2]. The target nucleic acid should be free of contaminants including protein, carbohydrate, lipids, or other nucleic acid, for example, DNA free of RNA or RNA free of DNA [3]. Quality and also integrity of the isolated nucleic acid will directly affect the results of all succeeding scientific research [4].

Since the first DNA isolation by Swiss physician Friedrich Miescher in 1869, there have been several methods developed over the years [7]. Traditional methods for the isolation of nucleic acids are liquid-liquid extraction (LLE) methods which involve the use of phenol organic solvent mixture containing phenol and chloroform to extract cellular materials followed by precipitation of the nucleic acids with alcohol. These LLE methods, along with their modifications, such as the guanidinium thiocyanate-phenol-chloroform extraction, the alkaline extraction, and the CTAB extraction methods, are problematic as they are time consuming (e.g. require multiple extraction steps), require the use of toxic chemicals and often provide low yields of nucleic acid. Further, the purified nucleic acids can be contaminated with the organic solvents or alcohol, both of which interfere with downstream applications. The use of multiple solvents and time-consuming steps to recover DNA and to remove cellular interferences, such as polysaccharides, polyphenols, and other secondary metabolites, with the risk that DNA isolation can be the major bottleneck in the bioanalytical workflow. As a consequence, fast and cost-efficient DNA extraction protocols that yield high quality DNA are highly desired in the study of species' molecular genetics. It is clear that the liquid extraction methods can't meet the requirements.

Newer methods for the purification of NA are based on solid phase extraction (SPE). SPE allows quick and efficient purification compared to conventional LLE methods [8]. With SPE, the NA of interest is bound to a solid support depending on the pH and salt content of the buffer, while impurities such as proteins and other non-target nucleic acids are washed away. The purified nucleic acid of interest is then eluted from the solid support. Thus, many of the problems that are associated with LLE such as incomplete phase separation can be prevented.

Solid-phase purification is normally performed by using a spin column, operated under centrifugal force. The first solid phase purification methods were based on the use of silica. Silica materials such as glass particles, glass powder, silica particles, glass microfibers, and diatomaceous earth carriers have been used in combination with aqueous solutions of chaotropic salts to isolate NA. Methods for the purification of NA using other types of support materials such as modified silica and resins have also been developed.

Four key steps involved in solid-phase extraction are cell lysis, NA adsorption, washing, and elution [6]. After the sample has been degraded by using lysis buffer, the desired NA will absorb to the solid surface with the aid of high pH and salt concentration of the binding solution [9]. Other compounds, such as protein may have strong specific bond with the column surface as well. These contaminants can be removed in the washing step by using washing buffer containing a competitive agent [9]. For the elution step, TE buffer or water is introduced to release the desired nucleic acid from the column, so that it can be collected in a purified state [9]. Normally, rapid centrifugation, vacuum filtration, or column separation is required during the washing and elution steps of purification process.

A mixed-bed solid phase nucleic acid extraction and its use in the isolation of nucleic acid have been disclosed [10]. The mixed-bed solid phases are the mixtures of at least two different solid phases, can be solid or semisolid, porous or non-porous. Each solid phase can bind to the target nucleic acid under different solution conditions and release the NA under similar elution conditions [10].

An alternative to the spin column is SPE based on the magnetic beads, which is a simple and efficient way for the purification of NA nowadays. Often, magnetic carriers with immobilized affinity ligands or prepared from biopolymer showing affinity to the target NA are used for the isolation process. For example, magnetic particles that are produced from different synthetic polymers, biopolymers, porous glass or magnetic particles based on inorganic magnetic materials such as surface-modified iron oxide [11]. Particles having magnetic or paramagnetic properties are encapsulated in a polymer such as magnetizable cellulose. The magnetic component of cellulose can also be substituted by other magnetic compounds such as ferrous oxide or nickel oxide [12]. Magnetic beads do not require any organic solvents and eliminate the need for repeated centrifugation, vacuum filtration or column separation. Nucleic acid purification by using zirconia bead is another type of magnetic bead-based purification [13].

Solid-phase reversible immobilization paramagnetic bead-based technology has been utilized for a PCR purification system to deliver quality DNA. It requires simple protocol without centrifugation and filtration. PCR amplicons bind to paramagnetic particles which draw them out of solution, allowing contaminants such as dNTPs, primers, and salts to be rinsed away [14].

Methods using silica beads and silica resins can isolate DNA molecules for subsequent PCR amplification. However, these methods have associated problems. First, beads and resins are highly variable depending on how well they are packed and are thus hard to reproduce. Each loading of a micro-channel can result in a different amount of packing and thus change the amount of DNA that adsorbed to the channel. Furthermore, these methods result in a two-step manufacturing process.

Magnetic beads and spin column-based DNA extraction methods have demonstrated some advantages over conventional methods. However, they still need lengthy process with multiple steps involving absorption, washing and elution, etc. With the ever-present goals of speed and low cost, there is a continuing need for new materials and procedures for extracting DNA in less time and with fewer opportunities for operator intervention and error.

Further, there have been multiple efforts trying to add various chemical reagents into the samples to avoid the need of NA purification. Copious effort has been made in designing the composition of the buffer system. While some of those approaches were successful for certain applications, most of them were not so lucky.

### REFERENCES

1. Wink M. An Introduction to Molecular Biotechnology: Molecular Fundamentals, Methods and Application in Modern Biotechnology. Weinheim, Germany: Wiley-VCH; 2006.
2. Doyle K. The Source of Discovery: Protocols and Applications Guide. Madison, Wis, USA: PROMEGA; 1996.
3. Buckingham L, Flaws M L, Molecular Diagnostics: Fundamentals, Methods, & Clinical Applications. Philadelphia, Pa, USA: F. A. Davis; 2007.
4. Cseke U, Kaufman P B, Podila G K, Tsai C-J. Handbook of Molecular and Cellular Methods in Biology and Medicine. 2nd edition. Boca Raton, Fla, USA: CRC Press; 2004.
5. Kojima K, Ozawa S. Method for isolating and purifying nucleic acids. United State patent US 2002/0192667 A1, December 2002.
6. Brooks G. Biotechnology in Healthcare: An Introduction to Biopharmaceuticals. London, UK: Pharmaceutical Press; 1998.
7. Dahm R. Friedrich Miescher and the Discovery of DNA. Amsterdam, The Netherlands: Elsevier; 2004.
8. Esser K-H, Marx W H, Lisowsky T. Nucleic acid-free matrix: regeneration of DNA binding columns. BioTechniques. 2005; 39(2):270-271.
9. Gjerse D T, Hoang L, Hornby D. RNA Purification and Analysis: Sample Preparation, Extraction, Chromatography. 1st edition. Weinheim, Germany: Wiley-VCH; 2009.
10. Smith C E, Holmes D L, Simpson D J, Kayzhendler J, Bitner R H, Groseh J C. Mixed-bed solid phase and its use in the isolation of nucleic acids. United State patent US 2002/0001812 A1, Promega Corporation, January 2002 or U.S. Pat. No. 6,376,194 B2, Apr. 23, 2002.
11. Berensmeier S. Magnetic particles for the separation and purification of nucleic acids. Applied Microbiology and Biotechnology. 2006; 73(3):495-504.
12. Nargessi R D. Magnetic isolation and purification of nucleic acids. U.S. Pat. No. 6,855,499 B1, Cortex Biochem, Inc., February 2005.
13. Applied Biosystems. MagMAX™ Total Nucleic Acid Isolation Kit. Foster City, Calif, USA: Applied Biosystems; 2008.
14. Beckman Coulter, Inc. Agencourt® AMPure® System: PCR Purification System. Chaska, Minn, USA: Beckman Coulter; 2009.

### SUMMARY OF THE DISCLOSURE

In general, the invention provides new and improved methods, systems and kits for rapid isolation of NA from biological samples containing cells. After lysis of the cells, NA is isolated through a membrane which is fabricated by incorporating solid sorbent, whose surface has been modified with different properties, into adhesives and processed to form the proper thickness and pores.

In one aspect, the invention features a method for the rapid isolation of NA, including: a) collecting biological samples containing cells and resuspending them in an aqueous buffer; b) incubating the resultant mixture with a lysis/denaturation solution to lyse the cells and denature DNA; c) neutralizing the mixture with a renaturation solution to generate a renatured mixture of dissolved plasmid DNA and flocculants containing insoluble genomic DNA and cellular debris; d) loading the renatured mixture directly to a (spin) column or syringe without first removing the flocculants from the mixture, which column having a filtration membrane; e) passing loaded sample mixture through the column such that the flocculants are packed on top of the filtration membrane while DNA passes through the membrane to be collected. This is a one-step filtration process of pushing the lysis solution containing NA directly passing through a membrane, or the so-called nucleic acid direct pass (NADP™) process. The key principle of this one-step NADP™ purification methodology involves the use of special materials in the membrane to eliminate certain ingredients, such as surfactants, proteins, and lipids that may interfering with subsequent applications. It allows to collect almost all of the NA present in the sample, especially for smaller NA.

The new NADP™ process is simple, convenient, fast and economical without the need of adding any reagents or changing the composition of the solution. It does not require special conditions, long exposure times, costly chemicals, or specialized equipment. The NADP™ process can be accomplished by using a simple DNA extraction device that is disposable after each use.

For most applications, nucleic acids obtained after the NADP™ process can be used directly. However, due to the complexity of biological cells, it may be necessary to restrict the presence of certain specific compounds in the lysis reagents in certain embodiments of the invention to suit the need of subsequent applications. Certain compounds, especially those that have similar chemical properties to that of nucleic acids, may pass through the membrane as well. Some of those compounds may result in unwanted consequence, such as being inhibitory to PCR reactions. Fortunately, those compounds are not crucial to the lysis process and alternatives can always been found to replace them. For example, certain reducing agents, and non-detergent inorganic salts are optional and can be replaced by other compounds.

In a second aspect, when a concentration or clean up step is absolutely necessary, the invention allows the modification of the composition of the membrane and/or the addition of another extraction matrix, such as silica, underneath the membrane. The said extraction matrix can be in the form of loose sorbent or membrane. With the addition of the NA binding matrix, the procedure needs to add more steps: more steps are needed: f) washing the column with a wash solution to remove soluble impurities, if any; and g) eluting NA from the column with an elution buffer. Actually, it has been found that by introducing the NADP membrane on top of the absorption matrix, there is no need to remove flocculants containing cellular debris prior to loading the (spin) column. Instead, the flocculants stay on top of the NADP filtration membrane throughout the purification process and do not interfere with subsequent wash or elution of the NA. Thus, the washing step can be omitted and only the elution step is really needed. This is called the NADP+™ process.

In addition, the extracted NA can be further separated into RNA and DNA by using either precipitation with organic solvent, such as isopropanol, or column technologies. Proteins that are trapped on top of the NADP filtration membrane can be harvested easily and cleaned with dialysis or other related technologies.

These and other features, embodiments, and advantages of the invention will be apparent from the description that follows.

### BRIEF DESCRIPTION OF THE DRAWINGS

FIG. 1. NA-Filter columns for NADP™ process: (A) open column with flat bottom; (B) flat bottom column filled with NADP membrane ( [symbol] ); (C) flat bottom column filled with NADP membrane and secured with O-ring ( [symbol] ) as shown in D.; (E) open column with conical bottom; (F) conical bottom column filled with NADP membrane on top of a porous sintered polyethylene or polypropylene supporting frit ( [symbol] ); (G) conical bottom column filled with NADP membrane with supporting frit and secured with O-ring.

FIG. 2. NA-Filter columns for NADP+™ process: (A) flat bottom with adsorbent powders ( [symbol] ) on top of a supporting frit; (B) flat bottom with an additional membrane ( [symbol] ) embedded with adsorbent powders inside without the use of a supporting frit; (C) conical bottom with adsorbent powders on top of a supporting frit; (D) conical bottom with an additional membrane embedded as a membrane with a supporting frit.

FIG. 3. Centrifuge column includes (A) a filtration column for centrifuge tube, (B) centrifuge tube suitable for outside of a filtration column (C); (D) flat bottom filtration basket for spin column; (E) conical bottom filtration basket for spin column; (F) collection tube for spin column.

FIG. 4. An overview of a 96 well plate-based NA filtration device (top view) as defined in ANSI_2_2004.

FIG. 5. NADP+™ assembly consisting of a NA-Filter column containing NADP membrane plus a SPE column containing silica.

FIG. 6. Application process of single barrel DNA filtration process. A. a syringe with lysis solution and E. Coli sample; B. swirling of the mixture; C. push liquid passing through the membrane and collection of liquid in Eppendorf tube.

FIG. 7. Slab gel electrophoresis of nucleic acids extracted from the E. Coli samples with RNA and DNA marked.

FIG. 8A. Gel electrophoresis results of DNA after PCR. The rightest column is the markers. The very next column to the markers was the PCR result of the original DNA extracted solution. Then, starting from right to left are the PCR results of solutions with sequential 10-fold dilutions of the original solution. The column immediately left of the original solution is 10-fold dilution. Both the original and the 10-fold dilution sample did not show any PCR results. This might be due to the fact that some impurities might be existing and the concentrations were too high. With more dilution, PCR was very effective for samples with 100 (Column 5), 1000 (Column 4), 10000 (Column 3), 100000 (Column 2) and 1000000 (Column 1) fold dilution. FIG. 8B. Slab gel electrophoresis of DNA samples from PCR along with markers (most right column). The concentrations of the samples from right to left are 10 million (10⁷), 100 million (10⁸), 1 billion (10⁹), 10 billion (10¹⁰), 10 billion (10¹¹), 1 trillion (10¹²), 10 trillion (10¹³) and 100 trillion (10¹⁴) fold dilutions.

FIG. 9. Gel electrophoresis of N gene and ORF gene extracted from E. Coli plasmid.

FIG. 10. Gel electrophoresis of DNA samples extracted from E. Coli plasmid with 10-million-fold dilution. A. Pass through the One-Step cartridge, B. Did not pass.

FIG. 11. Two steps linked NADP+™ process for viscous strawberry samples. C represent NADP column and CK represent commercial silica-based spin column. Each sample was run twice.

### DETAILED DESCRIPTION OF SELECTED EMBODIMENTS

The devices that are suitable for inclusion in the NA purification in the practice of this invention, examples include are consisted of 1) plastic tubes, weather in the form of a syringe barrel, spin column, regular column, or plates containing multiple tubes with two open endings, although other format and shapes will also function effectively; 2) a membrane, called NADP membrane, which consists of highly surfaced powders of sorbents glued together with adhesives and engineered to selectively prevent the pass through of specific species including but not limited to protein, lipid, debris of cells, etc. The sorbents may bare different functional groups on their surface to have particular properties for the selective absorption of impurities; 3) Alternatively, another sorbent matrix, which can be either in its loose form or also as a membrane, can also be used underneath the NADP membrane for the capture of NA passing through the NADP membrane. With all of those devices, there are some common optional accessories that may be used: 4) plunger, which is optional and can be replaced with a positive pressure or vacuum, that can push the liquid pass through the NADP membrane, and 5) stopper, which is also optional, that can be used at the end of the outlet tip to avoid dripping of liquid.

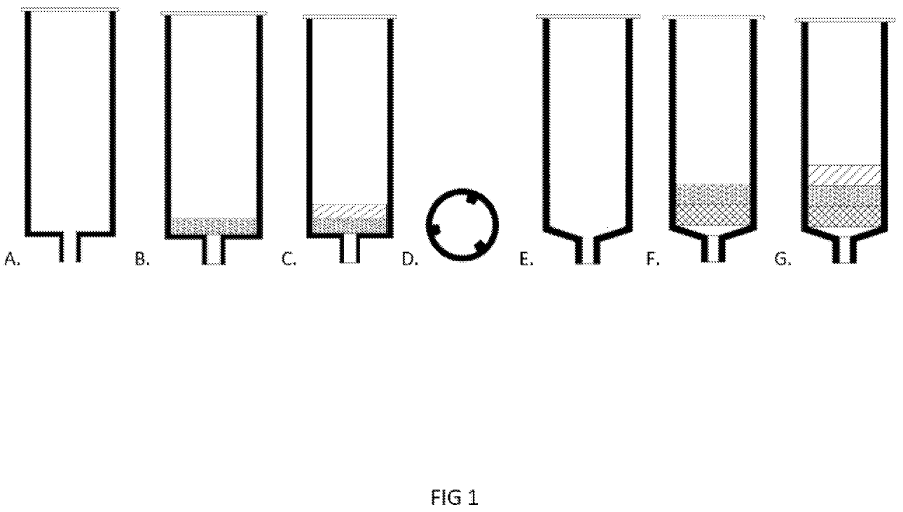

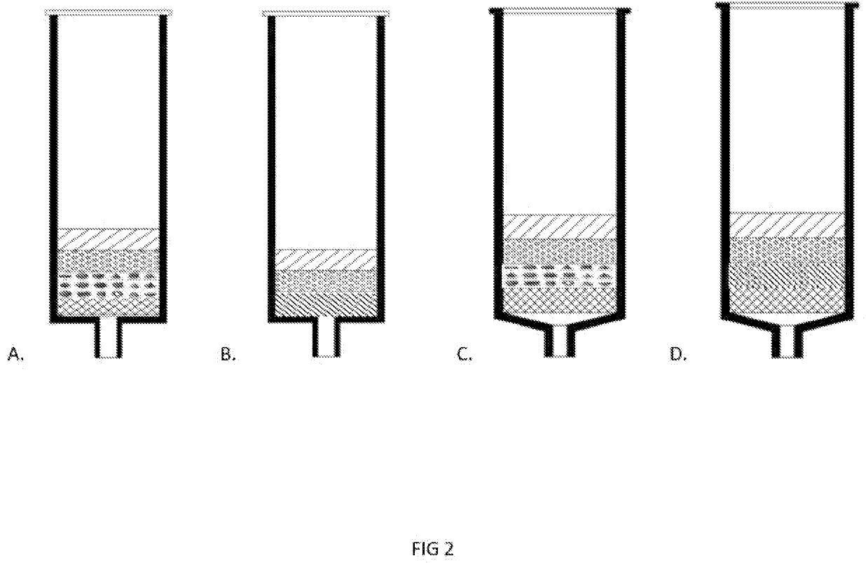

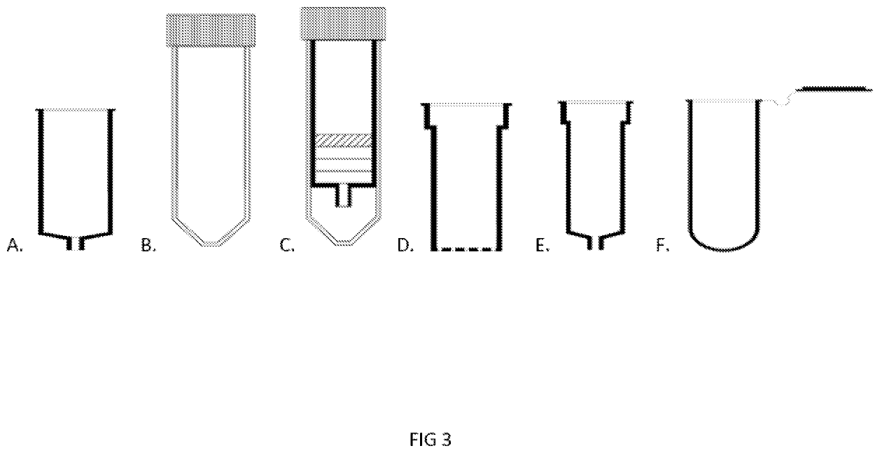

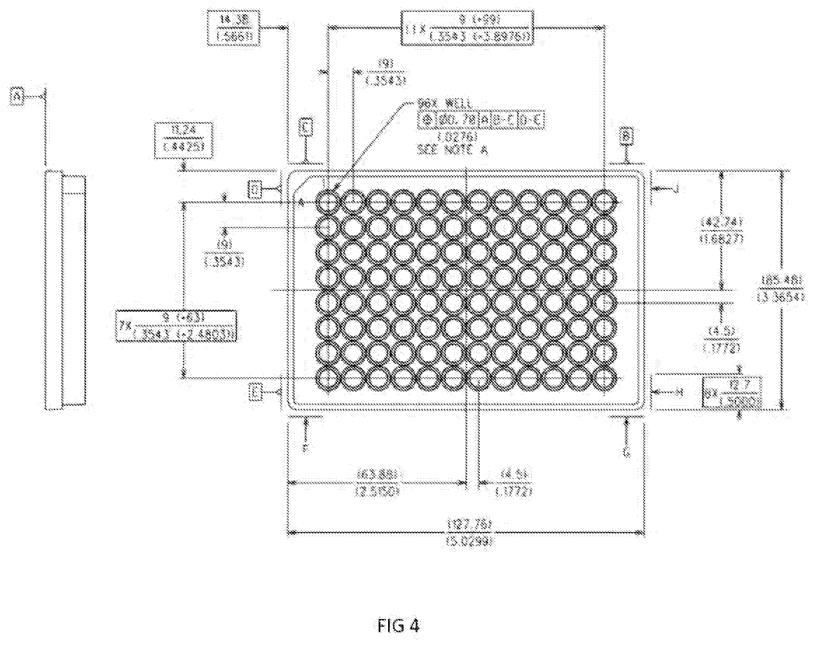

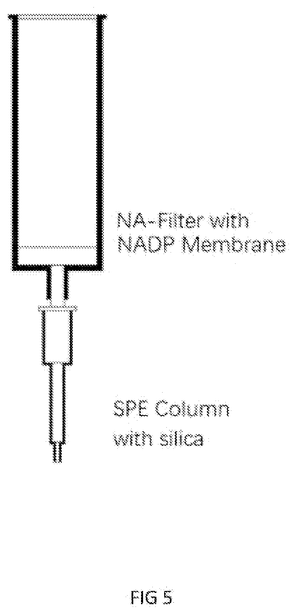

Under the same principle, the devices may adopt several formats like syringe, regular column, spin column, and even plates with multiple holes. All of these devices are used for filtration and are thus collectively called NA-Filters™. Based on their structure and outside appearances, they are classified as three categories with each of them having either the NADP™ or the NADP+™ process.
1) Columns: In the embodiment of a column for NADP™ process, such as syringe or regular chromatographic column, there is a big opening at the top for loading samples and a reduced exit at the lower end with a small tubing protrude out for easiness of collecting the eluent (FIG. 1A). The top edge may or may not have a small rim to extend towards outside for the conveniency of operation. A NADP membrane is placed on top of the plateau formed as a result of the reduced exit hole inside the column at the bottom (FIG. 1B). Then, a plastic O-ring is placed on the top to secure the NADP membrane in place (FIG. 1C).
   For most small columns, there is no need for extra support underneath the NADP membrane as long as the bottom is sufficiently flat. However, as the diameter gets bigger, a void volume may be needed underneath the membrane to allow the liquid to pass efficiently (FIG. 1D), thus, a conical bottom is used and a porous frit may be added first before the NADP membrane is added (FIG. 1E). Finally, an O-ring is added (FIG. 1F).
   In the embodiment of a NADP+™ column, all of the aforementioned NA-Filters can be used accordingly. The only difference is that an extract layer of absorbent has to be used. The adsorbent, such as silica, can be added into the flat bottom columns either directly as powder on top of a supporting frit (FIG. 2A) or as another membrane with adsorbent embedded inside the membrane and without the use of a supporting frit (FIG. 2B). For column with a conical bottom, the adsorbent powders can also be used on top of a supporting frit (FIG. 2C) or as an additional membrane on a supporting frit (FIG. 2D).
2) Centrifuge Column: another embodiment of the NA-Filter is centrifuge column, which comprise an internal filtration column (FIG. 3A) plus an outside tube (FIG. 3B) to collect liquid passing through the filtration column during centrifugation process (FIG. 3C). Depending on the bottom of the filtration column, the NADP membrane inside may adopt either FIG. 1C or FIG. 1G for flat bottom or conical bottom, respectively. They are sufficient for NADP™ filtration process.
   For NADP+™ process, the filtration column needs to add adsorption sorbents in addition to the NADP membrane. The adsorption sorbents can be added either as powder or as membrane. Therefore, the centrifuge filtration of column with flat bottom may adopt FIG. 2A and FIG. 2B for powder and membrane, respectively. Similarly, the centrifuge filtration of column with conical bottom may adopt FIG. 2C and FIG. 2D for powder and membrane, respectively.
   A special embodiment of the centrifuge column is called spin column, which has limited volume. The inner filtration column, whether flat bottom (FIG. 3D) or conical bottom (FIG. 3E), may hold less than 1 ml while the outside tube (FIG. 3F) is less than 1.5-2 mi. Similarly, the filtration columns may adopt similar configuration as in FIG. 1 and FIG. 2 for NADP and NADP+ process, respectively.
3) Plate: In another embodiment, the NA-Filter column may be part of a plastic plate, which may have multiple holes, often called wells, with internal structure similar to mini columns. The outside dimension of the plate has to follow certain international standard such as ANSI_SLAS_2_2004 (FIG. 4). The shape of the said wells can be either round or square and the number of the said wells can adopt the common numbers for these kinds of plates, such as 8, 12, 24, 48, 96, 384, 1536, etc. Within each of the wells, the column may adopt either the configuration in FIG. 1 or FIG. 2 for NADP or NADP+ process, respectively.
4) Two-pieces: For the NADP+™ process, in addition to using the NADP+ filtration unit, it is also possible to achieve the same objective by using a NADP filtration column followed with a regular SPE column, which may contain sorbent to adsorb NA, for the adsorption step. Alternatively, it is possible to link these two columns together to make a two-piece device for concentration purpose (FIG. 5).

Specifically, if the embodiment of a NA-Filtration column is a regular column, the volume of the column can be anywhere above 1 ml to a volume meeting actual need. In practice, the volume is preferably between 5-100 ml for preparative purpose.

A special embodiment of the NA-Filtration column is a syringe, the column sizes can be between 1 and 200 ml, preferably between 1 and 100 ml, and more preferably 1, 2, 3, 6, 10, 20, 30, and 60 ml etc. Actually, they can adopt any of the common syringes available on the market. For syringes, the plungers are readily available and are especially suited for point of care testing (POCT).

If the embodiment of the NA-Filtration column is a centrifuge column, the column sizes can be any volume between 1 ml and 200 ml, preferably 1-60 ml. A special centrifuge column is spin column whose volume is usually less than 2 ml. An outside column is simultaneously used to collect the liquid fractions filtered through the membrane at the bottom of the column.

When biological samples are placed inside the plastic columns, cell lysis solutions can be added and quickly mixed. Then, the solutions containing DNA (and/or RNA if present) can be forced to pass through the NADP membrane by a pressure generated by either a plunger, a gas pressure or a vacuum. The liquid passing through the NADP membrane can be collected for downstream application. For example, the liquid can be directly collected in PCR tubes and be amplified directly without any further treatment.

In general, the cell lysis may contain a variety of ingredients including enzymes, such as Proteinase K, and/or detergents including those that are denaturing, which are generally anionic or cationic, and those that are non-denaturing, which are generally either nonionic or zwitterionic. Denaturing detergents provide especially favorable results in many cases, and detergents that are either anionic or zwitterionic, particularly anionic, detergents, are of interest in many cases as well. Some of the more commonly used and highly convenient anionic detergents are SDS (C12), sodium octadecyl sulfate (C18), and sodium decyl sulfate (C10), particularly SDS. The amount of detergent can vary within the scope of the invention, although in most cases an amount within the range of from about 0.05% to about 0.5% by weight will be the most efficient. In many cases, amounts within the range of 0.1% to about 0.2% by weight will be even more efficient.

Selective absorption occurs when the lysis liquid pass through the NADP membrane, which provides the selectivity by the use of any of a wide variety of highly porous materials that have different properties. The base materials can be inorganic, such as silica oxide, zirconium oxide, or alumina oxide, to name a few. Further, the surface of those inorganic materials can be derivatized with various organic groups. Examples of the organic reagents can be neutral or ionic. Neutral groups can be a single ally group containing the number of carbons between one and any integer number, preferably between one and 30, and more preferably between one and eighteen. Neutral groups also include other ones, such as hydroxyl (OH), epoxy, and aromatic ones like phenyl, etc.; Ionic groups can be either cationic, such as the quaternary amine group, —NR₃⁺, where R can be an ally group having a number of carbon atoms between one and any integral number (C1-Cn), preferably between 1 and 30, and more specifically between 1 and 18. Other amines, such as primary amine like —NH₂, secondary amine like —NHR, tertiary amine as —NR₂ can be converted into cationic —NR₃⁺ group; or anionic, such as sulfonic (—SO₃⁻) or carboxyl (—COO⁻) group. With one or more of those organic groups, it is possible to offer different properties of the material surfaces and thus to selectively absorb different species inside the cell lysis buffer. In addition to inorganic materials, it is also possible to use organic polymers as the base materials.

Examples of the polymer materials may include but not limited to polystyrene-divinyl benzene (PS-DVB), acrylic materials, such as polyacrylic acid or polymethyl methacrylate (PMMA), poly allylamine, to name a few. On top of the base materials, it is also possible to attach various organic groups onto the surface making them baring different physical chemical properties.

One example of a NADP™ filtration column, which is used as the basic device to accomplish the procedure per the description above, is a syringe barrel, the volume of the syringe barrel can be any other volumes between 1 ml and 100 ml depending on the specific situation. When different sizes, such as 1, 2, 5, 6, 10, 30, 60, 100 ml of the syringe barrels are used, the range of the amounts of the absorption materials will change accordingly between 1 mg and 1000 mg, or between 10 and 100 for most of the barrels. For example, within a 3 ml syringe is a membrane composited from 100 mg mixed absorption materials, alternatively can be other amounts between 10-300 mg.

Another example is the NADP™ spin column, which allows the usage of centrifuge, if necessary, for NA purification. The volumes of the inner basket and outer tube in a spin column ranges from 0.5 ml to 60 ml, preferably 0.5-15 ml, and more preferably 0.8 ml and 2 ml for the inner and the outer, respectively. The amount of the absorption materials for the membrane inside the basket are between 1 mg-10 g, preferably 10 mg-1 g, and more preferably 15-500 mg depending on the sizes of the column.

Other examples of NADP™ filtration devices are multitube devices, 8, 12, 24, 48, 96 well plates with each well having a total volume between 1.0 ml to 2.2 ml. Within each well, membranes made up with any amounts between 10 and 200 mg, preferably between 10 and 100 mg of absorption materials can be placed. Alternatively, a 384 well plate can also be used with less absorption materials within each well. Even more (1536) and smaller wells, in theory, can be used, it is not practical to use those plates for the current applications.

Similarly, examples for NADP+™ filtration columns may adopt similar formats as syringes, columns, spin columns, and plates. Within each of them, one or more new sorbents with different properties as those used in the NADP™ membrane may be added underneath the NADP membrane to bring in new properties, such as NA capture or removal of specific ingredients, for example chlorophyll, in the liquid. The specific amount of the new sorbents will depend on the specific size of the column and the purpose of the intended use. Those sorbents can be added either as loose powders or as membrane.

The current invention brings in many advantages for NA purification. Some of the obvious ones are:
1. No equipment requirement: Among the advantages of the present invention are that NA purification using the DNA filtration devices described above can be performed easily, with no need of using complicated equipment. It can be done by using most of the common equipment in the lab. Even further, the procedure can be performed at any place without any specific requirements for laboratory equipment. This is especially suitable for remote area and POCT.
2. One step: A further advantage of the NA filtration devices described herein is that NA purification with these devices can be performed in a one-step procedure. After being added into the devices, the cell lysis solution can be pushed through the NADP™ membrane at the bottom of the device to be collected.
   This is a typical filtration process and is significantly different from the current SPE processes, which need NA capture (absorption), wash, and desorption steps as commonly seen in magnetic bead-based NA extraction method and the silica-based spin column method. Thus, separate steps for cell lysis and for precipitation of the liberated NA for example are not required in the filtration process. All such functions can be achieved by a single immersion of the cells in the NA extraction solution followed with a simple push of the liquid passing through the membrane. Agitation may be useful in certain cases, but mild agitation such as swirling or gentle stirring will generally suffice.
   As noted above, the extraction can be performed at mild temperatures, including ambient temperature. Even in the NADP+™ process, a second step of adding elution solution can still be accomplished simply adding some new solution.
3. Short time: A further advantage of the use of the NA filtration devices described herein is that NA extraction with these devices can be performed in a one-step procedure in a relatively short period of time compared to the corresponding procedures of the prior art. The NA filtration process with the use of the NA filtration devices is superfast with the whole process being completed as short as less than one minutes.
   Upon filtration, NA precipitation, if needed, herein can be achieved by adding NA absorbents within as little as one min and to less than ten minutes, often less than five minutes, and in many cases within a period of time ranging from about five seconds to about three minutes, or from about five seconds to about one minutes.
   Once precipitated, the DNA can be recovered by conventional means, examples of which are decantation, centrifugation, filtration, and spooling (drawing DNA as a precipitate onto a rotating glass stirring rod).
4. Economical: The procedure can also be performed without the use of numerous additional components that are commonly included in NA extraction solutions and procedures of the prior art. Substantially none of these components are present in this NA filtration process. Examples of these components are proteases, reducing agents, and non-detergent, inorganic salts. These components can be included if desired, but successful and effective results can be obtained without their inclusion. The expression “substantially none” or “substantially no” as used herein to describe the amounts of proteases, reducing agents, and non-detergent, inorganic salts, means that any amounts that are present are either trace amounts, amounts included unintentionally, and/or amounts that do not significantly affect the rapidity or completeness of the NA purification.
   Examples of proteases that are known for use in NA extraction and not needed in the practice of this invention are serine proteases, such as proteinase K, trypsin, chymotrypsin, elastase, subtilisin, streptogrisin, thermitase, aqualysin, plasmin, cucumisin, and carboxypeptidase A, D, C, or Y; cysteine proteases such as papain, calpain, or clostripain; acid proteases such as pepsin, chymosin, and cathepsin, and metalloproteases such as pronase, thermolysin, collagenase, dispase, aminopeptidases, and carboxypeptidase A, B, E/H. M. T. or U. Other known enzymes of similar utility will be apparent to those knowledgeable in NA extraction, and are likewise not needed. In fact, no enzymes of any kind are needed in the NA filtration solution. Examples of reducing agents that are known for use in NA extraction and not needed in the practice of this invention are dithiothreitol, B-mercaptoethanol, dithioerythritol, 2-aminoethanethiol, and 2-mercaptoethanol.
   Other known reducing agents of similar activity will be apparent to those knowledgeable in NA purification, and are likewise not needed. Examples of non-detergent, inorganic salts that are commonly used in NA extraction and not needed in the practice of this invention are sodium chloride, sodium acetate, and potassium chloride. Here as well, other similar salts will be apparent to those knowledgeable in NA extraction techniques, and are likewise not needed.
5. High throughout: This process of NA filtration with the use of multiple well based DNA extraction devices can be performed in parallel with multiple samples being processed simultaneously. The multiple wells can be any number of wells that is larger than one and is on a single plate. Examples included 8, 12, 24, 48, 96, 384 wells, to name a few. An alternative way to achieve parallel assay is by placing multiple single tubes in a suitable device that operate under such a condition that each and every tube has the same experience and condition.
6. Large quantity: The NADP™ filtration process can be scaled up as needed. Therefore, this process can be easily adopted to the preparation of DNA and RNA.
7. Applications: The DNA filtration devices and procedures described herein are useful for purifying NA from cells without cell walls, tissues (e.g., muscle) or organs (e.g. hair follicles) comprising such cells, and whole organisms (e.g., nematodes), including both epithelial and non-epithelial cells and tissues, and mammalian (e.g., human), non-vertebrate, and insect cells, both from primary and secondary (e.g., tissue culture) sources. Other applicable cell and tissue types include but are not limited to epithelial, muscle, connective, and nervous tissues and include squamous, cuboidal, columnar, and pseudostratified epithelial cells, endothelial cells, mesothelial cells, fibroblasts, neurons, blood cells, muscle cells, and stromal cells. These cells can be found, for example, in the skin, the lungs, blood vessels, pericardium, stomach, and intestines, as well as in exocrine tissue, the pancreas, kidney tubules, the nasal and bronchia passages, the uterus, and the Fallopian tubes.

Therefore, the NA filtration devices and procedures described herein are simple (one-step), fast (less than one minute), easy (no equipment requirement), convenient (suitable for point-of-care), economical (less solvent and instrument cost), high throughput (parallel), and broadly applicable (most kinds of cells). The following example is presented for illustrative purposes only.

### EXAMPLES

This invention primarily involves with the isolation of NA (DNA and RNA) from the mixture of cell lysates and other sources. It does not directly involve with the breakdown of cell walls and/or cell membranes. Therefore, it can be applied directly to any samples, such as plant, stool, vomit, saliva, and semen, etc. providing suitable lysis solutions are applied. For the same reason, this invention can be applied to other biological species such as E. Coli, virus, phage, etc.

However, by changing the composition of the lysis solution, it is possible to develop new lysis solution which is more compatible with the current invention and is more universally applicable.

Even further, by adding some more separation media to the bottom of the column, it is possible to provide the capability of separating DNA and RNA while isolating them from biological samples.

### Embodiment 1: E. Coli NA

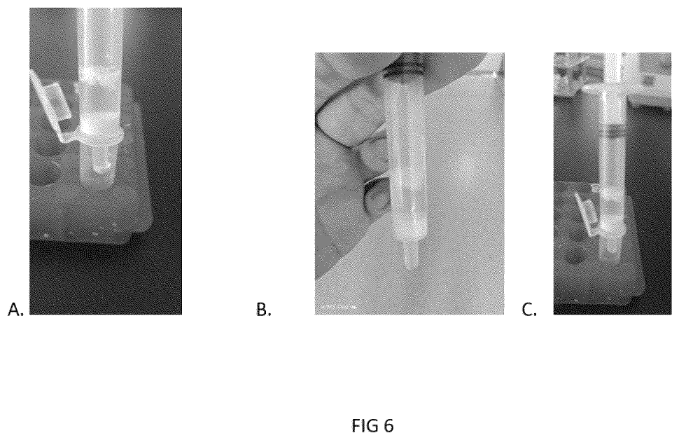

5 ml of E. Coli samples were added into a syringe with lysis solution using SDS instead of guanidine salt as the chaotropic and deproteinization agent (FIG. 6A). With gentle swirling (FIG. 6B), the liquid was pushed by a plunger for syringe through the NADP™ membrane sitting at the bottom part of the syringe. The liquid was collected in a 1.5 ml Eppendorf tube (FIG. 6A)

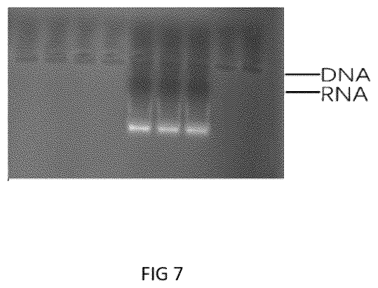

The extracted nucleic acids were analyzed by UV spectrometry and slab gel electrophoresis. The UV indicated that the concentration of NA in the extracted solution was about 2 OD, each OD equals about 33 μg/ml. Electrophoresis shows that both DNA and RNA were extracted out though this one-step process (FIG. 7).

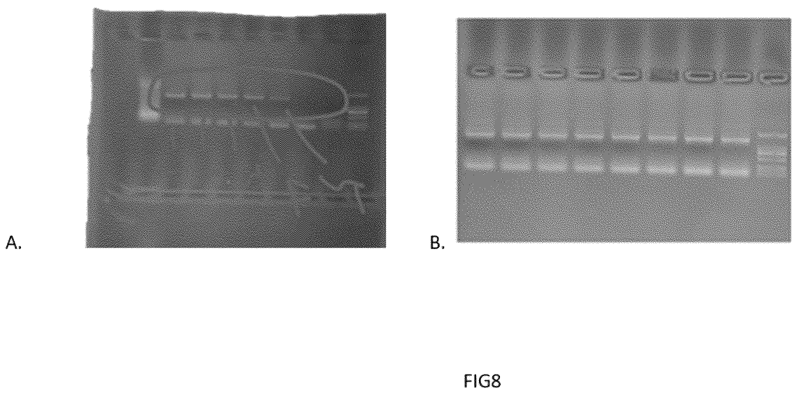

In addition, we also used PCR to demonstrate the feasibility of the extracted DNA for PCR reaction. We used QuanStudio™ 3D digital PCR to run the assay. The PCR process is as following: 1)95° C. 5 min; 2) 95° C. 30s; 3) 52° C. 30s; 4) 72° C. 55s; 5) go to 2) for a total of 40 cycles, and finally 6) 72° C. 2 min. The results are shown in FIG. 8A. Since the sample with one-million-fold dilution still showed very strong PCR signal in gel electrophoresis, we performed more dilution using the sample of one-million-fold dilution before we conduct PCR. To our surprise, we could still see the PCR results even after 7 consecutive 10-fold dilutions (FIG. 8B).

### Embodiment 2: Covid-19 Genes

Extraction of DNA from E. Coli plasmid containing N gene and ORF gene, which may serve as a positive control of Covid-19 samples because those genes can be derived from NA in Covid-19 virus. The basic procedures are: 1) colony recovery: select 10 μl each frozen bacteria containing N gene and ORF gene and coat them onto the culture plate containing ampicillin antibiotics. Take the streaking method to obtain a single colony strain; 2) small colony culture: pick a single colony strain and add it to the liquid medium containing ampicillin antibiotics, and cultivate for 12 hours at 37° C., 200 rpm constant temperature incubator; 3) colony collection: collect the cultured positive bacteria in a 1.5 ml centrifuge tube; 4) colony plasmid extraction: follow the extraction steps to extract two bacterial species plasmids.

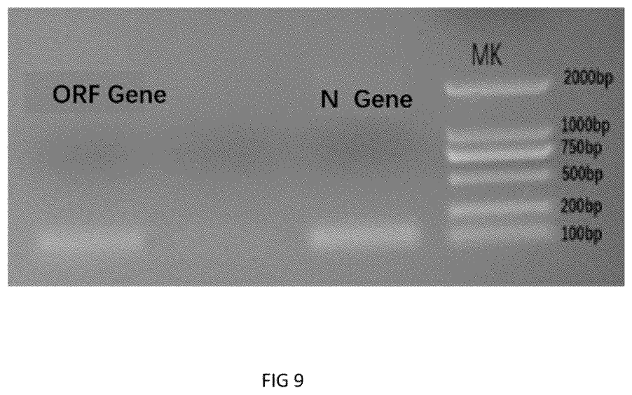

For PCR amplification of plasmid containing N gene ORF gene, primers listed in Table 1 were used. The details of the PCR system are listed in Table 2. The sequence of the PCR is as following: 1) 95° C. 2 min, 2) 95° C. 20 sec, 3) 95° C. 20 sec, 4) 95° C. 20 sec, 5) Go to step 2 for 40 cycles; 6) 95° C. 2 min. The results of the extracted DNA for N gene and ORF gene are shown in FIG. 9. The number of copies, concentration and fragment sizes of the genes extracted from the plasmid are obtained with 13 dilutions of PCR products with each time using 50 μl to be added into 450 μl deionized water. The final results are listed in Table 3.

TABLE 1
Primers

| Name of Primers | Sequence of Primers |
|---|---|
| Covid-19-N-F | GGGGAACTTCTCCTGCTAGAAT |
| Covid-19-N-R | CAGACATTTTGCTCTCAAGCTG |
| Covid-19-ORF-F | CCCTGTGGGTTTTACACTTAA |
| Covid-19-ORF-R | ACGATTGTGCATCAGCTGA |

TABLE 2
PCR system

| Name of the system | Quantity (50 μl system) |
|---|---|
| Hotstart DNA Polymerase | |
| 10xPCR Buffer | |
| 2.5 mM dNTP | 5 |
| PRIMER-F | 3 |
| PRIMER-R | 5 |
| Plasmid | 2 |
| Deionized Water | Up to 50 μl |

TABLE 3
Outcome of N gene and ORF gene purification by NADP ™ Filter

| Name of the gene | Product Concentration | Fragment size |
|---|---|---|
| N Gene | 466.7 ng/μl | 121 bp |
| ORF Gene | 359.5 ng/μl | 116 bp |

Using the following formular:

Number of copies=6.02×10¹⁴×fragment concentration/324×fragment size

it is possible to calculate that the numbers of N gene and ORF gene were 7.1×10¹⁴ and 5.7×10¹⁴, respectively.

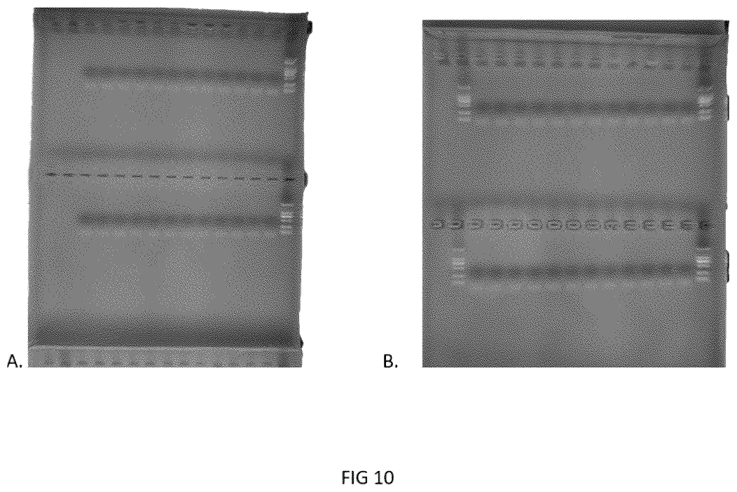

In the meantime, we chose the sample after sever times (10⁷-fold) dilution to compare the DNA recovery by passing part of the sample through the one-step NADP™ filtration cartridge while another part did not. Gel electrophoresis data indicated that there was almost no difference between the original sample vs. the one passed through the cartridge (FIG. 10). FIG. 10 indicates that there is little difference between the sample passing or not passing through the one-step cartridge. Therefore, it is necessary to use a more sensitive and more quantitative method to determine the difference.

For more accurate comparison, we diluted the original extraction samples 10-fold and run the quantitative PCR. The results are listed in Table 4, which indicates that the C(t) values for non-filtered and the cartridge filtered samples are 20.64+0.69 (RSD 3.36%, n=6) and 21.53+0.61 (RSD 2.81%, n=6), respectively. It can be concluded that there is very little loss of DNA after passing through the cartridge. Thus, it can be concluded that this one-step cartridge is a very effective method for extraction of DNA and the loss of DNA in passing through the membrane is very low.

TABLE 4
qPCR results for samples passing vs. not passing NADP membrane

| Sample C(t) | 1 | 2 | 3 | 4 | 5 | 6 | Average |
|---|---|---|---|---|---|---|---|
| C(t)-no cartridge | 21.02 | 21.9 | 20.56 | 19.74 | 20.07 | 20.56 | 20.64 |
| C(t)-cartridge | 22.24 | 22.2 | 21.22 | 20.83 | 20.8 | 21.89 | 21.53 |

### Embodiment 3: Two Steps Linked NADP+™ Process for Viscous Samples

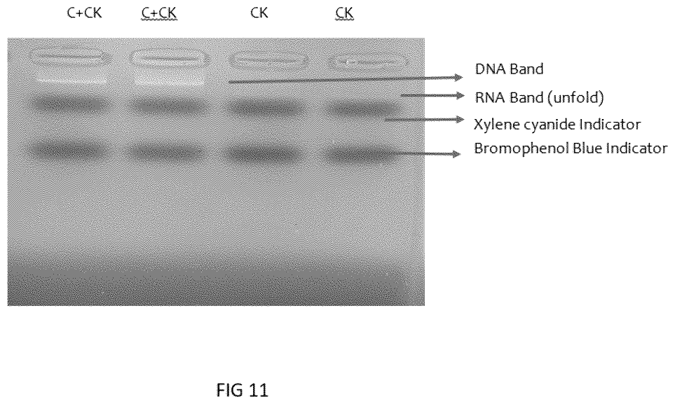

Some examples like strawberry, the lysis solution after cell lysis is very viscous and sticky. Common DNA extraction process does not work well. In this case, it is advantages to use a two-step process. For example, when commercial spin column CK was used for extraction of DNA, no DNA was extracted out (FIG. 11). However, if a NADP™ spin column is used as the first step and followed up with the CK column, DNA can be extracted well. Therefore, NADP™ process not only can be used as one-step DNA extraction method, it can also be used as a pre-treatment to facilitate the overall efficiency of DNA extraction.

### Embodiment 4

NADP™ and NADP+™ processes can also be applied for the extraction of NA from blood and other biological matrixes. Especially, it is useful in extraction of cell free DNA and/or circular tumor DNA. Cell-free DNA (cfDNA) is a liquid biopsy marker that can carry signatures (i.e., mutations) associated with certain pathological conditions. Therefore, the extraction of cfDNA from a variety of clinical samples can be an effective and minimally invasive source of markers for disease detection and subsequent management. In the oncological diseases, circulating tumor DNA (ctDNA), a cfDNA sub-class, can carry clinically actionable mutations and coupled with next generation sequencing or other mutation detection methods provide a venue for effective in vitro diagnostics. However, cfDNA mutational analyses require high quality inputs. This necessitates extraction platforms that provide high recovery over the entire ctDNA size range (50→150 bp) with minimal interferences (i.e., co-extraction of genomic DNA), and high reproducibility with a simple workflow. In common SPE process, it is hard to purify small DNA fragments as they are harder to be absorbed, easier to be washed away during the purification process. In NADP™ process, small fragments of DNA can easily pass through the NA filtration membrane.

In the claims appended hereto, the term “a” or “an” is intended to mean “one or more.” The term “comprise” and variations thereof such as “comprises” and “comprising.” when preceding the recitation of a step or an element, are intended to mean that the addition of further steps or elements is optional and not excluded. All patents, patent applications, and other published reference materials cited in this specification are hereby incorporated herein by reference in their entirety. Any discrepancy between any reference material cited herein or any prior art in general and an explicit teaching of this specification is intended to be resolved in favor of the teaching in this specification. This includes any discrepancy between an art-understood definition of a word or phrase and a definition explicitly provided in this specification of the same word or phrase.

What is claimed:

1. A NADP membrane comprising of porous sorbent materials embedded within a polymeric adhesive and processed through unique engineering process.

2. The said porous sorbent materials in claim 1 can be inorganic materials like silica, zirconium oxide, or alumina oxide, diatomaceous earth, glass fiber, magnetic and other materials that are modified chemically to have different characteristics.

3. The said porous sorbent materials in claim 1 can also be made of polymeric materials, such as polystyrene-divinyl benzene (PS-DVB), polymethyl methacrylate (PMMA), poly allylamine, with different organic groups on the surface to have different physical chemical properties.

4. The said adhesive of claim 1 can be, based on for specific applications, any one or more kinds of the following classes of adhesives, urethanes, epoxies, resins, cyanoacrylates, fluoropolymer, and methacrylate.

5. A device for purifying nucleic acid (NA) from cells with or without cell walls, comprising of: a column having a column with both ends having diameters being between 0.1 and 20 cm with preference between 0.2 and 10 cm, length being between 1 and 100 cm with preference between 0.5 and 20 cm.

6. The upper (sampling) end of said column of claim 5 may have a flat shoulder around or any other embodiment, such as screw graves, which are not critical for the said process but may be necessary for the convenience of handling or covering of the column.

7. At the lower (collection) end of said column of claim 5 may have either a flat bottom of the same diameter with a net (FIG. 1A), a reduced diameter with a hole in the middle and a stage formed between the hole and the wall of the column (FIG. 1B), or a conical bottom with a cone volume (FIG. 1C), a short tubing with reduced diameter, which can be any size smaller than that of the original column with preference of being similar to that of a typical syringe barrel, is extended out for liquid to exit and can be covered with an optional rubber cap.

8. The said membrane of claim 1 comprising of the materials embedded within inert materials to form a uniform disc is placed inside the column and secured, with a plastic O-ring, on top of the said flat bottom or the said conical bottom with the support of a plastic frit to cover the exit hole.

9. The said column of claim 5 can be made of plastics, rubber, glass, porcelain or any other materials that are resistant to corrosion of common chemical reagents and not causing potential contamination to the content inside with preferred materials being plastics such as polypropylene (PP) or polyethylene (PE).

10. The said column of claim 4 may adopt the format of a plate having 8, 12, 24, 48, 96, and 384 holes as defined in the American National Standard Institute (ANSI), such as ANSI/SLAS 1-2004(R2012) for 96-well plate footprint dimensions.

11. The said column of claim 5 wherein the said column is an inner part, which has a matching outside tube with a larger diameter plus a cap to keep the inner column suspending on the top of the outer tube leaving some room at the bottom for storage of liquid passing through the membrane.

12. A so called NADP process for extracting NA from cells with or without cell walls comprising of immersing biological samples containing cells in an aqueous cell lysis solution inside the said column of claim 5 for about five seconds to about 10 min at an ambient temperature, to cause NA to be liberated from said cells followed by recovering the said NA by either pushing from the top or pulling from the bottom the lysis solution through the NADP membrane.

13. The said lysis solution in the said NADP process of claim 12 comprises of detergents, buffer, with or without protease, reducing agent, and inorganic salt with the detergents being a member selected from the group consisting of anionic and zwitterionic detergents, such as sodium dodecyl sulfate (SDS), sodium octadecyl sulfate, and sodium decyl sulfate at a concentration from about 0.1% to about 0.2% by weight.

14. The said NADP process of claim 12 is a one-step processing with the NA from the sample along with the lysis solution to be filtrated out in about 5 seconds to about 5 minutes, preferably from about 5 seconds to about 2 minutes.

15. Wherein the NADP process of claim 12, lysis solution in the column may be subjected to a positive gas pressure from the top, a negative vacuum from the bottom, or a plunger inside to drive the liquid pass through the membrane to be collected for subsequent use.

16. Wherein the NADP process of claim 12, the NA samples can be any size present in the biological samples with the preference to be smaller sizes of NA, such as circular free DNA, etc.

17. The said NADP process of claim 12 can be extended with an additional step to use an elution buffer to elute NA absorbed on to the absorption materials underneath the NADP membrane and thus constituted as the NADP+ process.

18. Wherein the NADP+ process of claim 16, the said absorption materials can be any solid materials that absorb NA, such as silica.

19. Wherein the NADP+ process of claim 16, the said elution buffer can be any solvent, such as a basic aqueous solution, that can break the binding between NA and the absorption materials.
